(19) United States
(12) Patent Application Publication
(10) Pub. No.: US 2009/0249685 A1
(43) Pub. Date: Oct. 8, 2009

(54) Closed loop biomass energy system

(76) Inventors: Troy D. Flowers, Bargersville, IN (US); Timothy C. Helfeman, Indianapolis, IN (US); Stephen J. Hogan, Indianapolis, IN (US)

Correspondence Address: KRIEG DEVAULT LLP, ONE INDIANA SQUARE, SUITE 2800, INDIANAPOLIS, IN, 46204-2079, US

(21) Appl. No.: 12/383,656

(22) Filed: Mar. 27, 2009

Related U.S. Application Data
(60) Provisional application No. 61/040,465, filed on Mar. 28, 2008.

(51) Int. Cl. C10L 5/46; C02F 1/469; C10B 53/08; C10B 47/00; C10B 51/00; C10B 57/10; C12M 1/00

(52) U.S. Cl. 44/605; 204/648; 202/204; 202/84; 210/170.08; 210/770; 204/553; 201/19; 44/629; 435/289.1

(57) ABSTRACT

A closed loop biomass energy system includes at least one agricultural facility that produces agricultural waste; a waste processing system operable to convert the waste into biomass; a wastewater treatment system operable to treat wastewater created by the waste processing system and an energy production system operable to utilize the biomass to produce energy and provide the energy to an energy end-user. In one form, the energy is steam and the energy end-user is a biofuel production facility that utilizes the steam to power production of a biofuel. In a further aspect of this form, wastewater created as a byproduct of the biofuel production is transferred to and treated by the wastewater treatment system. However, additional forms, embodiments, features and aspects are contemplated as discussed in further detail in this document.

### CROSS-REFERENCE TO RELATED APPLICATIONS

The subject application claims priority to U.S. Provisional Application No. 61/040,465 filed Mar. 28, 2008, the contents of which are incorporated herein by reference in their entirety.

### BACKGROUND

Agricultural facilities can produce large amounts of agricultural waste. If this agricultural waste is not handled properly, it can potentially present significant environmental harm, including for example soil and groundwater pollution and air contamination. Even under some current handling practices, environmental damage can still result from the agricultural waste. For example, agricultural facilities commonly use mechanical scraping or flush systems to convey waste into a centralized collection pit. This waste typically contains 2% to 6% solids, and may be further processed by a slope screen separator, a vertical screw press or a roller press. These processes generally recover less than 50% of the total solids. The resulting liquid waste is diverted to waste separation lagoons. While the recovered solids are typically either land applied or windrow composted to produce bedding, water recycled through waste separation lagoons is often associated with objectionable odors, releasing powerful greenhouse and toxic gases, and representing a source of surface and ground water contamination.

Some types of agricultural waste can be used to provide a biomass as a feasible fuel source. For example, dry dairy waste provides an estimated 9,500 BTUs per pound. However, systems and methods for sufficiently drying the waste to provide economically viable amounts of energy are currently lacking. Thus, there remains a need of addressing the manner in which agricultural waste is handled in order to reduce environmental harms and enhance its feasibility for use as a source of energy.

### SUMMARY

The present application concerns closed loop biomass energy systems and their use in handling agricultural waste and providing energy. More particularly, in one embodiment, a system for providing energy from agricultural waste includes at least one agricultural facility from which the agricultural waste is provided and a waste processing system coupled with the at least one agricultural facility to receive the agricultural waste. The waste processing system is generally structured and operable to remove water from the agricultural waste to produce a biomass. The system also includes an energy generation system coupled with the waste processing system to receive the biomass, and is structured to utilize the biomass to produce a quantity of energy. The energy is provided to at least one energy end-user coupled with the energy generation system to receive and utilize the quantity of energy. The system also includes a wastewater treatment system coupled with the energy end-user and the waste processing system to receive wastewater therefrom and treat the wastewater to provide treated water to at least one of the at least one agricultural facility, energy end-user, energy generation system and waste processing system.

In one form of this embodiment, the energy generation system is structured to utilize the biomass to produce a quantity of energy in the form of steam, electricity or both. In a further aspect of this form, the at least one energy end-user includes a manufacturing facility, such as a biofuel facility, that utilizes steam from the energy generation system to power a production process, such as the production of a biofuel.

In another embodiment a system includes at least one agricultural facility operable to produce agricultural waste and a vacuum conveyance system coupled to the at least one agricultural facility and operable to deliver the agricultural waste to a waste processing system. The waste processing system is generally operable to remove water from the agricultural waste to provide a biomass. The system also includes an energy generation system operable to utilize the biomass to produce energy and provide the energy to the at least one agricultural facility. In one form of this embodiment, the agricultural waste is electro-coagulated and decanted at least once by the waste processing system. In another form of this embodiment, the system further includes a wastewater treatment system coupled to the waste processing system and operable to treat the water removed from the agricultural waste.

In yet another embodiment, a method includes collecting agricultural waste from at least one agricultural facility; removing water from the agricultural waste to provide a biomass and a first portion of wastewater; utilizing the biomass to produce a quantity of energy; providing the energy to at least one energy end-user; treating the first portion of wastewater to provide useable water; and transferring the useable water to the at least one agricultural facility. In a further form of this embodiment, the at least one energy end-user is a manufacturing facility and the method further also includes powering production processes of the manufacturing facility with the quantity of energy. In one aspect of this form, the production processes generate a second portion of wastewater which is treated to provide useable water that is transferred to the at least one agricultural facility. In yet another form of this embodiment, removing water from the agricultural waste includes electro-coagulation of the agricultural waste. In one aspect of this form, the removing also includes decantation of water from the agricultural waste following the electro-coagulation.

In still another embodiment, a method for preparing a biomass includes providing agricultural waste; electro-coagulating the agricultural waste to separate solids in the agricultural waste from water in the agricultural waste; and removing the solids from the agricultural waste to provide the biomass and a quantity of wastewater. In one form of this embodiment, the removing includes decanting said agricultural waste. In another form of this embodiment, the removing also includes drying the solids to reduce residual water therein. In yet another form of this embodiment, the providing includes collecting the agricultural waste with a waste vacuum system from at least one agricultural facility.

Still, further embodiments, forms, features, aspects, benefits, objects, and advantages of the present invention shall become apparent from the detailed description and figures provided.

### BRIEF DESCRIPTION OF THE DRAWINGS

FIG. 1 is a flow diagram illustrating one form of a closed loop biomass energy system.

FIG. 2 is a flow diagram illustrating an alternative embodiment closed loop biomass energy system.

FIG. 3 is a flow diagram illustrating vacuum conveyance and waste processing systems of the systems illustrated in FIGS. 1 and 2.

FIG. 4 is a plan view of the vacuum conveyance system set forth in FIG. 3.

FIG. 5 is a plan view of a valve pit of the vacuum conveyance system illustrated in FIGS. 3 and 4.

FIG. 6 is a plan view of a vacuum station of the vacuum conveyance system illustrated in FIGS. 3 and 4.

FIG. 7 is a perspective view of a thermal drying system of the waste processing system set forth in FIG. 3.

FIG. 8 is a flow diagram of a wastewater treatment system of the systems illustrated in FIGS. 1 and 2.

FIG. 9 is a plan view of the wastewater treatment system of FIG. 8.

FIG. 10 is a flow diagram of an energy generation system of the systems illustrated in FIGS. 1 and 2.

FIG. 11 is a diagrammatic plan view of a fluidized bed thermal oxidizer of the energy generation system of FIG. 10.

FIG. 12 is a flow diagram of the energy end-user of the system illustrated in FIG. 1.

### DETAILED DESCRIPTION OF REPRESENTATIVE EMBODIMENTS

For the purposes of promoting an understanding of the principles of the invention, reference will now be made to the embodiments illustrated in the drawings and specific language will be used to describe the same. No limitation of the scope of the invention is intended. Any alterations and modifications in the described embodiments, and any further applications of the principles of the invention as described herein are contemplated as would normally occur to one skilled in the art to which the invention relates.

The present application is generally directed to systems and methods for utilizing agricultural waste to produce energy. More particularly, in one form, agricultural waste provided by at least one agricultural facility in the form of animal by-products, such as manure, is processed by a waste processing system to reduce at least a portion of the moisture or water content in the agricultural waste. In one non-limiting example, the moisture or water content is reduced by in-line maceration, electro-coagulation, decanting, and/or drying, amongst other possible alternatives. After the moisture or water content is reduced or at least partially eliminated, the agricultural waste provides a biomass that is used to fuel an energy generation system, along with wastewater. The energy generation system can use the biomass to produce energy through combustion, gasification or pyrolysis, just to name a few possibilities. Moreover, the energy can be provided in any suitable form, including steam or electricity, although other forms of energy are contemplated.

The energy is then provided to at least one energy end-user, which may be the at least one agricultural facility, a manufacturing facility, a power-grid or any combination thereof, just to provide a few non-limiting examples. The system also includes a wastewater treatment system that is coupled with the waste processing system and receives the wastewater therefrom. The wastewater treatment system treats the wastewater to provide useable water that is transferred to at least one of the at least one agricultural facility and the at least one energy end-user, although it is contemplated that the useable water could be transferred to any party having a need thereof. In one particular form, the wastewater treatment system utilizes a treatment method that creates an algae biomass as a byproduct of the wastewater. In this form, the algae biomass can be provided to the energy generation system and used with the biomass derived from the agricultural waste to create energy. Additionally, in one form where the at least one energy end-user includes a manufacturing facility, wastewater from the manufacturing facility can also be treated by the wastewater treatment system. Moreover, in one particular form of the manufacturing facility, such as a biofuel facility, carbon dioxide is created and can be transferred to the wastewater treatment system and used to promote growth of the algae biomass. Still, additional features and aspects of the present application will be discussed below.

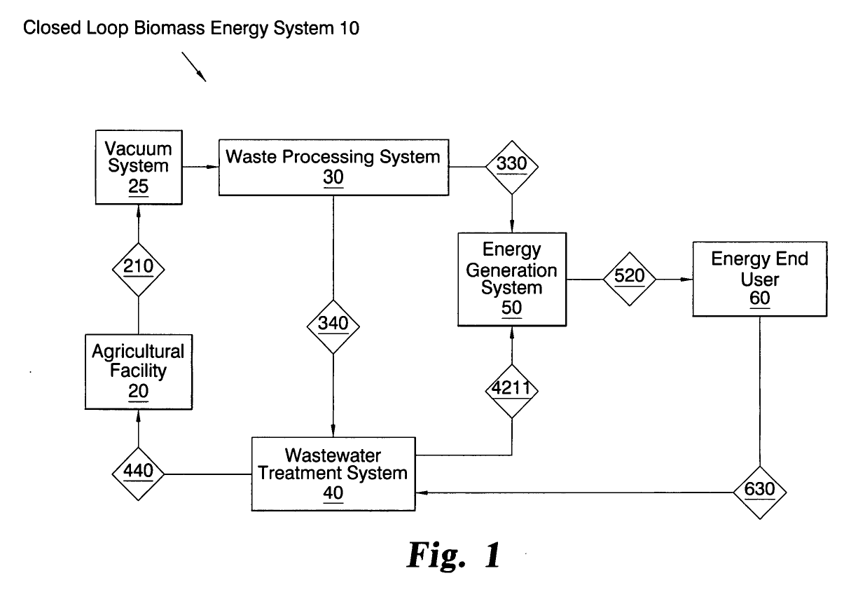

FIG. 1 is a flow chart illustrating one embodiment of a closed loop biomass energy system 10. Closed loop biomass energy system 10 includes at least one agricultural facility 20, waste vacuum conveyance system 25, waste processing system 30, wastewater treatment system 40, energy generation system 50, and energy end-user 60. Agricultural facility or facilities 20 are operable to create agricultural waste 210 and may be, for example, livestock farms such as dairies, hog farms, feed lots, chicken farms and/or turkey farms, just to provide a few possibilities. As would be appreciated by those skilled in the art, the form of agricultural waste 210 will depend on the type of agricultural facility 20. In one form for example, agricultural waste 210 can be animal products, such as manure from animals kept at the at least one agricultural facility 20. In one more particular, non-limiting form, agricultural waste 210 is cow manure that has been removed from the at least one agricultural facility 20 with water. Still, other forms for agricultural waste 210 are contemplated.

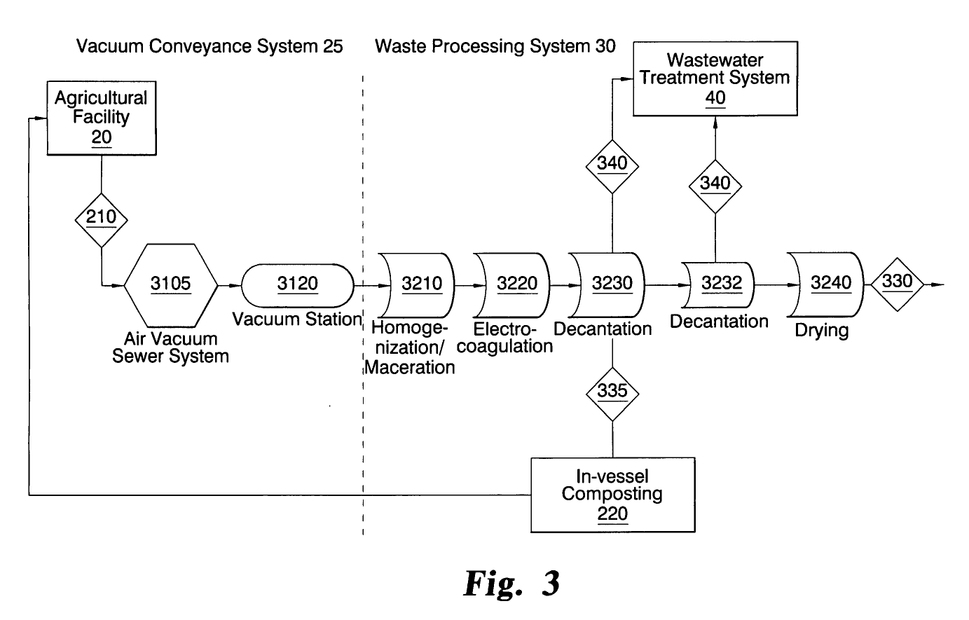

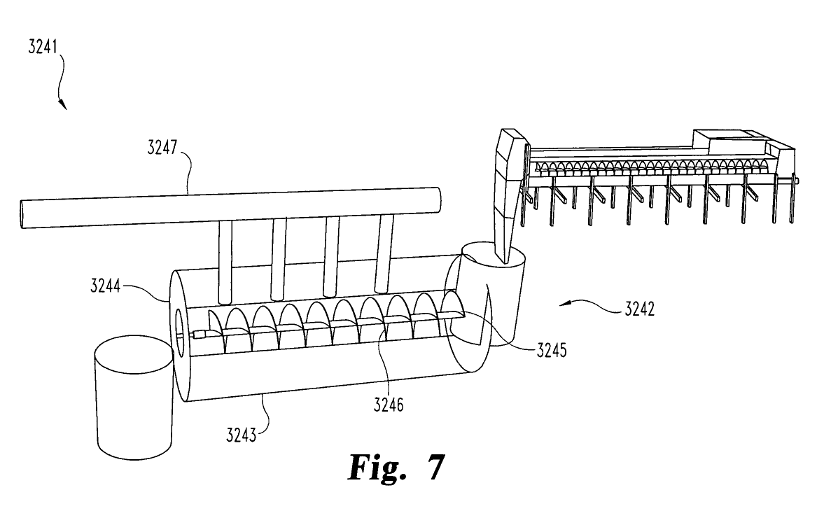

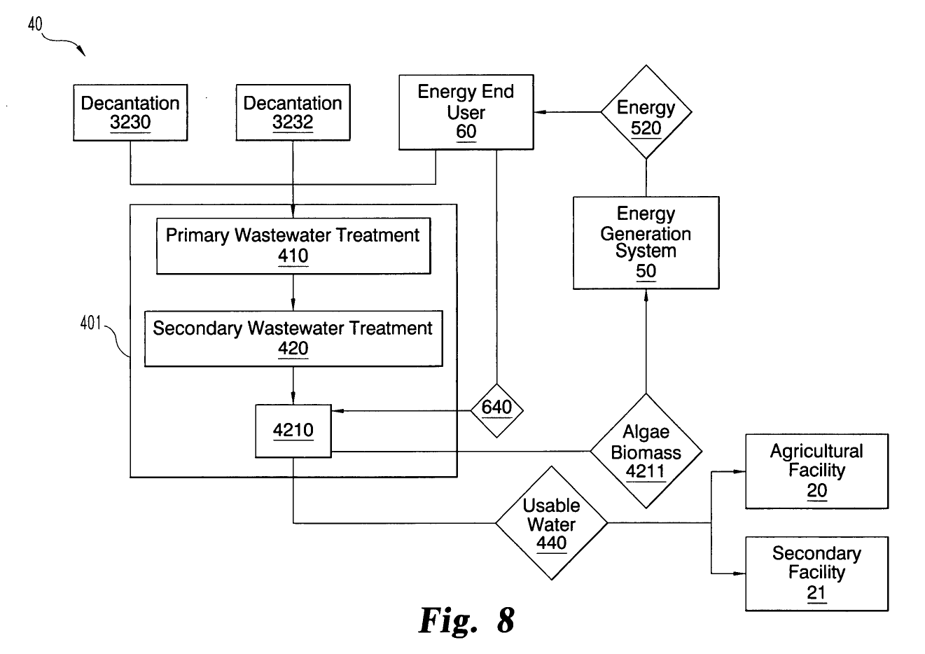

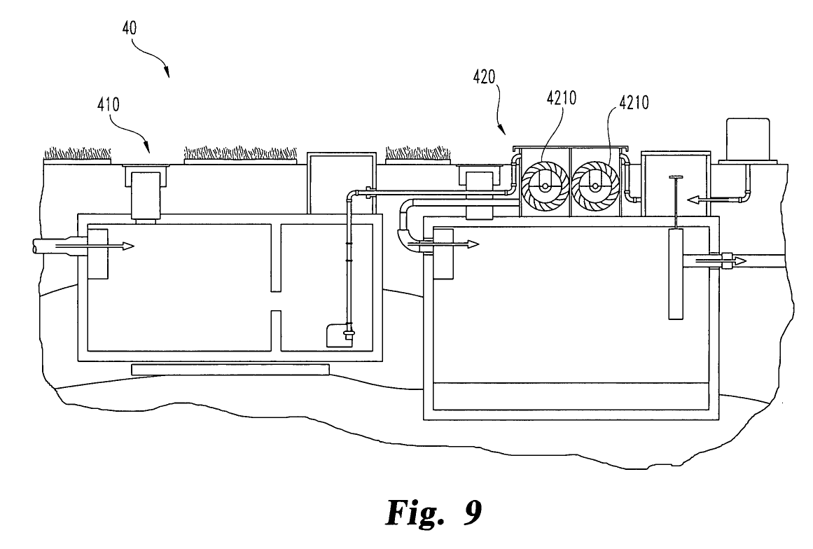

Waste vacuum conveyance system 25 is coupled with the at least one agricultural facility 20 and waste processing system 30 and is operable to transfer agricultural waste 210 from the at least one agricultural facility 20 to waste processing system 30. Waste processing system 30 is generally operable to at least partially dry agricultural waste 210 to provide a biomass 330 consumable by energy generation system 50, further details of which will be described below in connection with FIGS. 3 and 7. Wastewater treatment system 40 is operable to treat wastewater received from waste processing system 30 and provide useable water, further details of which will be provided below in connection with FIGS. 8-9.

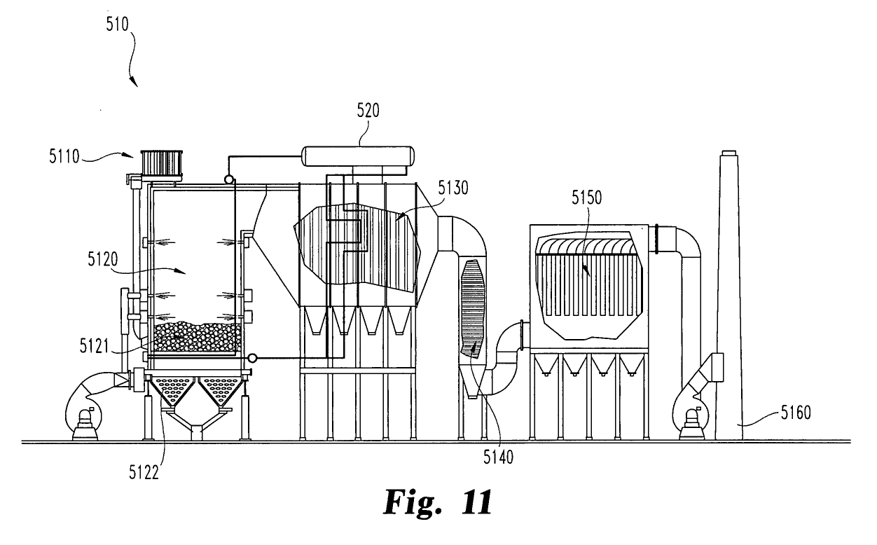

Energy generation system 50 is generally structured to utilize biomass 330 from waste processing system 30 to fuel production of energy 520. In one form, energy generation system 50 can include a thermal combustor system or a pyrolythic system. As would be appreciated by those skilled in the art, the form of energy 520 may vary with the form of equipment employed by energy generation system 50 for using biomass 310 to fuel production of energy 520. As non-limiting examples, energy generation system 50 may provide energy 520 in the form of steam, electricity and/or mechanical power, such as a drive shaft or other type of mechanism for transferring mechanical power, just to name a few possibilities. In one particular form, energy generation system 50 includes a fluidized bed thermal oxidizer 510 which produces energy 520 as steam, further details of which will be provided below in connection with FIG. 11. Still, it should be appreciated that other forms for energy generation system 50 are contemplated. In the illustrated embodiment of FIG. 1, energy end-user 60 is a manufacturing facility, such as a biofuel production facility like an ethanol plant or a biodiesel plant, although it should be appreciated that energy end-user 60 can generally be any party having a need for energy 520 provided by energy generation system 50. For example, in alternative embodiments, energy end-user 60 could be one of the at least one agricultural facility 20 and/or a power grid.

During operation of system 10, agricultural waste 210 from the at least one agricultural facility 20 is conveyed to waste processing system 30 by vacuum conveyance system 25 to be processed off-site by waste processing system 30. Waste processing system 30 converts agricultural waste 210 into biomass 330 and wastewater 340. Biomass 330 is sent to energy generating system 50 as fuel for creating energy 520. Wastewater 340 is sent to wastewater treatment system 40 and is further processed to provide algae biomass 4211 to be consumed by energy generation system 50 and usable water 440 to be returned to the at least one agricultural facility 20 and/or provided to one or more secondary facilities. Energy generation system 50 generates energy 520 to be used by energy end-user 60. As indicated above, in system 10 energy end-user 60 is in the form of a manufacturing facility, and it creates wastewater 630 which is sent to wastewater treatment system 40 for processing.

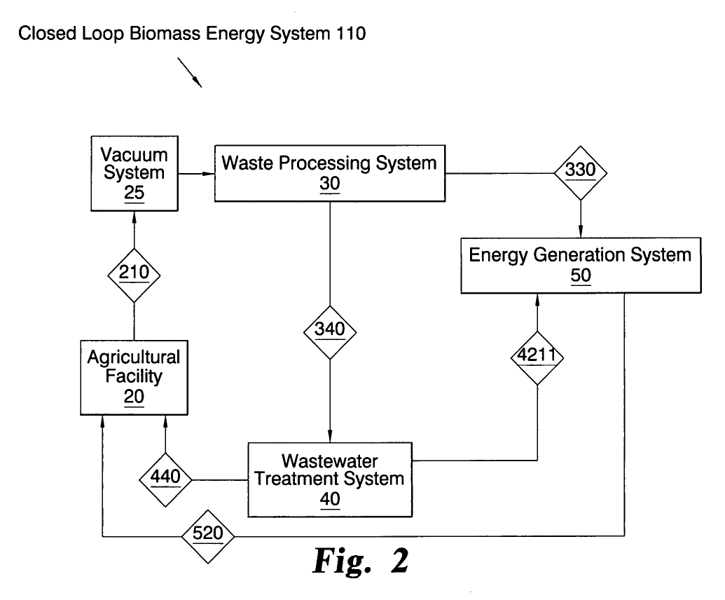

An alternative embodiment biomass energy system 110 is illustrated in FIG. 2, where like numerals refer to like features. System 110 includes at least one agricultural facility 120, a vacuum system 25, a waste processing system 30, a wastewater treatment system 40 and an energy generation system 50. Similar to system 10, agricultural waste 210 is provided by the at least one agricultural facility 120 and is conveyed to waste processing system 30 by vacuum conveyance system 25 to be processed off-site by waste processing system 30. Waste processing system 30 converts agricultural waste 210 into biomass 330 and wastewater 340. Biomass 330 is sent to energy generating system 50 as a combustible fuel. Wastewater 340 is sent to wastewater treatment system 40 and is further processed to provide algae biomass 4211 to be consumed by energy generation system 50 and usable water 440 to be returned to the at least one agricultural facility 120 and/or provided to one or more secondary facilities. Energy generation system 50 generates energy 520 that is used by the at least one agricultural facility 120 and/or can be provided to one or more secondary sources.

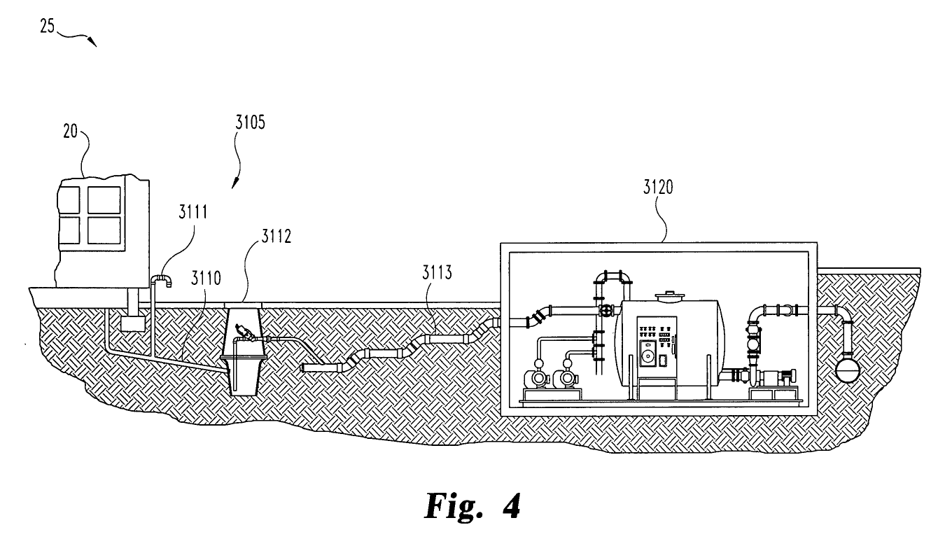

With reference to FIGS. 3-7, the interaction between the at least one agricultural facility 20, 120, vacuum conveyance system 25 and waste processing system 30 of systems 10, 110 is illustrated in further detail. More particularly, vacuum conveyance system 25 of systems 10, 110 conveys agricultural waste 210 using an air vacuum sewer system 3105 to waste processing system 30. While not illustrated, it is contemplated that agricultural waste 210 can be conveyed from the at least one agricultural facility 20, 120 to the waste processing system 30 as a liquid slurry. In one form, vacuum conveyance system 25 may include an AirVac system manufactured by AirVac, Inc. of 4217 North Old U.S. 31, P.O. Box 528, Rochester, Ind. 46975. As illustrated in FIGS. 4-6, air vacuum sewer system 3105 includes valve pit 3112, main waste line 3113, and air-intake 3111. In at least one form, valve pit 3112 is a centralized collection pit fluidly coupled to drain lines from each of the at least one agricultural facility 20, 120. Other forms of the present application contemplate valve pit 3112 being fluidly coupled to a collection pit at each of the at least one agricultural facility 20, 120.

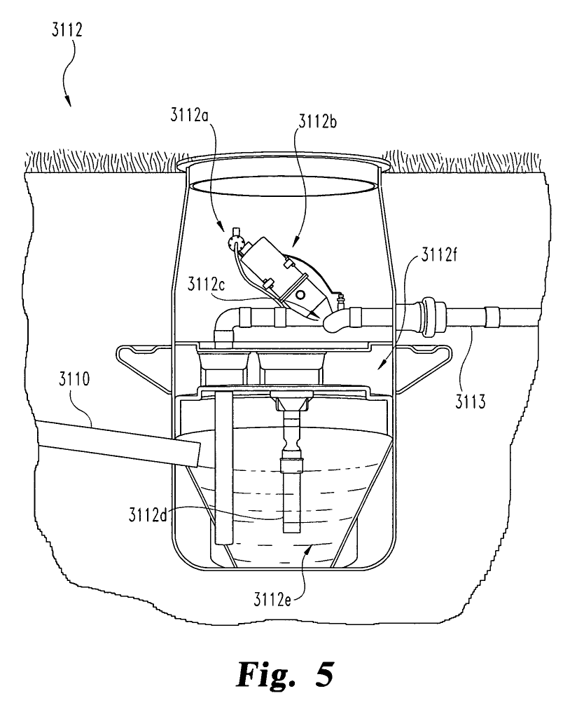

As shown in FIGS. 4 and 5, valve pit 3112 serves as the entry point for agricultural waste 210 from the at least one agricultural facility 20, 120 to enter air vacuum sewer system 3105, flowing via gravity through line 3110 into a sealed sump 3112e. Line 3110 includes air-intake 3111 through which atmospheric air used for transport enters line 3110. Generally, odors at this air-inlet are limited due to the small volumes of sewage and short detention time in sealed sump 3112e. Valve pit 3112 is controlled with a valve assembly 3112b. As agricultural waste 210 accumulates, air trapped inside a sensor pipe 3112d pushes in a diaphragm (not shown) in valve assembly's 3112b controller/sensor unit (not shown), signaling a valve (not shown) to open. Preferably, when a predetermined amount of agricultural waste 210 accumulates in sealed sump 3112e, the valve automatically opens. The air pressure differential propels agricultural waste 210 toward vacuum station 3120. In one embodiment, velocities of about 15-18 ft/sec (4.5 to 5.5 m/s) are reached. It is contemplated that these aid to liquefy agricultural waste 210 while it is being transported through main waste line 3113 to vacuum station 3120. The valve in valve assembly 3112b stays open for a predetermined period of time during this cycle. One form of the present application contemplates the valve in valve assembly 3112b staying open for a period of 4-6 seconds. Other forms of the present application contemplate the valve in valve assembly 3112b staying open for periods shorter or longer than 4-6 seconds.

Valve pit 3112 also includes a valve pit cone 3112c, a pit bottom 3112f, and an in-sump breather unit 3112a. The valve pit cone 3112c houses the vacuum valve and controller/sensor unit. The valve pit cone 3112c is fabricated with filament-wound fiberglass and is generally suitable for H-20 traffic loading. The pit bottom 3112f separates the upper and lower chambers of valve pit 3112 to prevent water from entering sealed sump 3112e and is bolted to sealed sump 3112e by stainless steel hardware (not shown) and a sealing O-ring (not shown). The in-sump breather unit 3112a, in the event of low vacuum conditions where the valve would not open, contains floats to protect the controller/sensor unit from unwanted liquid because the controller/sensor unit relies on closing valve assembly 3112b.

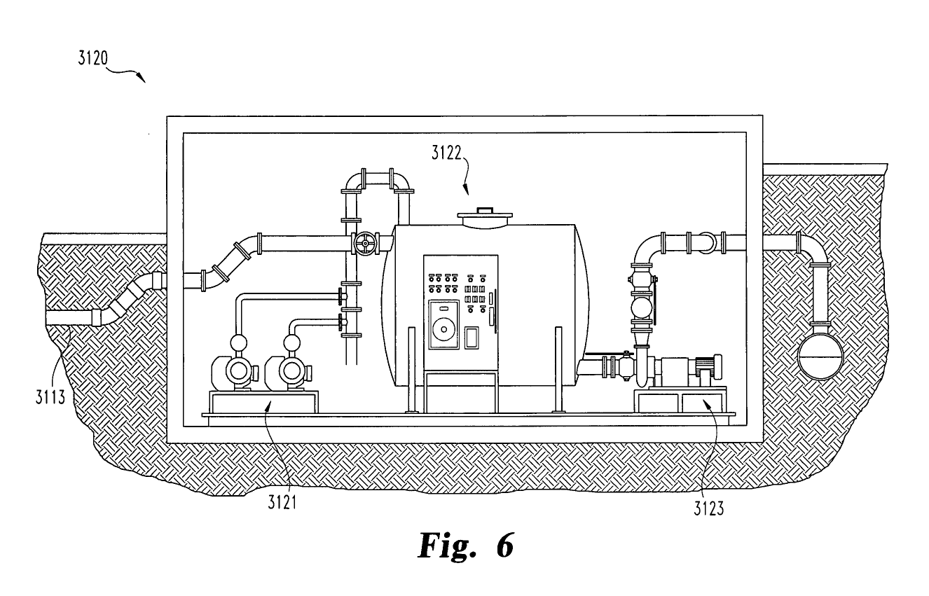

Referring now to FIG. 6, vacuum station 3120 includes vacuum pumps 3121, at least one collection tank 3122, and sewage pumps 3123. Vacuum pumps 3121 maintain the system vacuum in a preset operating range. In one form, the preset operating range is about 16″ to about 20″ mercury vacuum (Hg) (−0.5 to −0.7 bar). Vacuum pumps 3121 need not run continuously; rather the operation is cyclical. In one form, when both vacuum pumps 3121 are used, it is contemplated that each will run between 2 to 3 hours per day, although other values are contemplated. In one form, as agricultural waste 210 and atmospheric air enter vacuum sewer system 3105 during valve cycles, the vacuum on vacuum sewer system 3105 will gradually decrease from about 20″ to about 16″ Hg. Vacuum pumps 3121 are sized to bring the vacuum level back to about 20″ Hg within a predetermined time. In one form, the predetermined time is about 3 minutes, although other values are contemplated. Without being limited to any particular form, it is contemplated that the vacuum pumps 3121 could be 10, 15 and/or 25 horsepower (7.5, 11 and 18.7 Kw). One form of the present application contemplates vacuum pump 3121 having a rotary vane configuration; however, other pump configurations may be used without deviating from the scope of the present application.

Sewage pumps 3123 preferably include at least two, non-clog, dry-pit, horizontal, centrifugal sewage pumps each capable of pumping the peak flow of vacuum conveyance system 25. Other forms of the present application contemplate sewage pumps 3123 which are dry pit submersible pumps. Collection tank 3122 may be formed from either steel or fiberglass and is sized to include a volume suitable for accommodating peak activity of vacuum conveyance system 25. By way of a non-limiting example, collection tank 3122 has a volume from about 1000 to about 6000 gallons (3.8 to 22.7 m³). When a single valve pit 3112 is utilized, a single waste line 3113 connects to collection tank 3122. In other forms where multiple valve pits are utilized, several waste lines 3113 will connect to collection tank 3122. Alternatively, the several waste lines 3113 may converge before collection tank 3122, and a single line 3113 will connect with collection tank 3122. In one alternative form of the present application, vacuum conveyance system 25 may convey agricultural waste 210 to a geographic high point, from which gravity flow sewers (not shown) will deliver agricultural waste 210 to waste processing system 30.

It should be appreciated that alternative systems may be utilized for conveying agricultural waste 210 to waste processing system 30, such as conventional pumping or gravity conveyance. However, while not intending to be bound to any theory by which the present system achieves its affect, vacuum conveyance may be utilized because it can readily convey agricultural waste 210 up a gradient, and also reduce the amount of maintenance required for conventional pumping systems.

Referring again to FIG. 3, agricultural waste 210 is conveyed from the at least one agricultural facility 20, 120 by vacuum conveyance system 25 to waste processing system 30.

Waste processing system 30 is generally operable to remove water from or dry agricultural waste 210 and includes several processes by which water and/or moisture are removed from agricultural waste 210 to produce biomass 330 and wastewater 340. It should be appreciated that waste processing system 30 may utilize processes in addition to or in lieu of those described below, and may also utilize fewer processes for removing water or moisture from agricultural waste 210 than what is illustrated in FIG. 3. Efficient dewatering of agricultural wastes 210 represents an important and challenging component of processing agricultural waste 210 to provide biomass 330. Dairy waste typically contains mucus not generally found in other animal wastes, and this mucus can create problems for traditional dewatering systems. In one exemplary form of the present application, it has been determined that efficient pre-treatment is necessary to achieve certain dewatering objectives, including capturing in excess of 99% of total suspended solids, and producing cake moisture of 65% or lower. By achieving these goals, biomass 330 becomes a viable biomass fuel source or a suitable raw material for in-vessel composting. In addition, wastewater 340 separated from biomass 330 will contain a much lower concentration of solids and nutrient concentrations, making it more readily treatable and recyclable by the wastewater treatment system 40.

More particularly, as will be described below, agricultural waste 210 is subjected to several processes at waste processing system 30. However, it should be appreciated that in alternative forms waste processing system 30 may utilize processes for drying agricultural waste 210 in addition to or in lieu of those described below and/or may utilize less than all of the processes described for drying agricultural waste 210. In the illustrated form, processing system 30 includes homogenization and in-line maceration 3210, electro-coagulation 3220, decantation 3230, second decantation 3232, and drying 3240. In one alternative form, waste processing system 30 may utilize the addition of a polymer to agricultural waste 210 between decantation 3230 and second decantation 3232. If used, polymer addition involves automatically metering in a quantity of any of a variety of polymers at a calculated rate based on the solids content of agricultural waste 210 after processing by decantation 3230. As would be appreciated by those skilled in the art, applying the polymer before second decantation 3232 reduces the amount of polymer required compared with adding the polymer before decantation 3230.

In one form, first decantation 3230 and second decantation 3232 each utilizes serial decantation. As would be appreciated by those skilled the art, serial decantation utilizes enhanced gravitational forces to separate products of differing densities and offer high throughput, continuous flow operation, small footprint, low maintenance, and minimal oversight operation. After undergoing decantation 3230 at least a portion of agricultural waste 210 in the form of dried waste 335 is diverted to in-vessel composting 220 instead of proceeding to second decantation 3232. One form of the present application contemplates diverting about 25-50% of agricultural waste 210 to in-vessel composting 220. In-vessel composting 220 facilitates the production of bedding materials for possible use by the at least one agricultural facility 20, 120.

Homogenization/maceration 3210 of agricultural waste 210 through agitation and in line maceration improves decantation 3230 efficiency. It is also contemplated that agricultural waste 210 may be subject to dilution and acid treatment to improve dewatering capacity. Pre-treatment of agricultural waste 210 through the application of a direct electric current or electro-coagulation 3220 breaks colloidal suspensions and emulsions in aqueous solutions. The mucus found in agricultural waste 210 is a colloidal material. Electro-coagulation 3220 causes solids to form hydrophobic conglomerates that can be separated from the water. Electro-coagulation 3220 also destroys bacteria and other single cell organisms, eliminating the source of much of the odor associated with agricultural waste 210.

Electro-coagulation 3220 involves passing agricultural waste 210 between at least two metal plate electrodes. The electrical current causes solids to conglomerate and separate from the water, allowing for a more efficient decantation 3230.

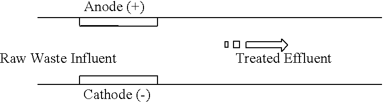

With minimal operational costs, electro-coagulation 3220 represents a cost effective pre-treatment that facilitates subsequent dewatering through decantation or through a variety of other mechanical dewatering approaches, including but not limited to pressing or filtering. Application of electro-coagulation 3220 to animal wastes reduces biochemical and chemical oxygen demand (BOD and COD), metals, ammonium, Phosphorous, total suspended solids and dissolved solids. It is further contemplated within the scope of the present application that the use of electro-coagulation 3220 allows for efficient decantation 3230 without the subsequent use of polymers while also facilitating subsequent wastewater treatment.

Other forms of the present application contemplate using a horizontal screw process in addition to or in lieu of decantation 3230. As would be appreciated by one skilled in the art, screw presses utilize a screw augur to force wastes through a screen. Solids capture rates are highly dependent on the particle size profile of the suspended solids and screw presses work best with a more concentrated waste stream. Still other forms of the present application contain using dewatering techniques involving filtration including micro, ultra, and/or nano filtration.

Additionally, an Internal Drum Thickener (IDT) and screw presses are contemplated for use in addition to or in lieu of decantation 3230, 3232 in other forms of the present application. An IDT is a rotating drum screen system which offers a high solids capture rate with low energy consumption. One form of an IDT is manufactured by Press Technology & Manufacturing, Inc. and is designed to thicken stock and sludges. Typically, agricultural waste 210 will enter the IDT at a projected solids concentration of about 4% to 7% and exit the IDT at a projected solids concentration of about 10% to 12%. This thickening provides an effective pre-treatment to facilitate further processing by second decantation 3232. When utilized, one form of a horizontal screw press contemplated by the present application includes an Agri-Press™ Screw Press manufactured by Press Technology & Mfg., Inc. having an address of 1315 Lagonda Avenue, Springfield, Ohio, 45503. The Agri-Press™ Screw Press is able to achieve total solids concentrations ranging from about 38% to 43%.

It is contemplated that drying 3240 could be any process suitable for reducing residual water in agricultural waste 210 after second decantation 3232. In one non-limiting example, drying 3240 utilizes a thermal drying system, further details of which are provided in connection with FIG. 7 in which thermal drying system 3241 is illustrated. Thermal drying system 3241 has an enclosed auger system 3242 that transects a highly insulated hot-box 3243. Agricultural waste 210 augers down the length of hot-box 3243 within housing 3244. Housing 3244 heats up to vaporize water in agricultural waste 210 and to volatilize some fraction of the volatile composition of agricultural waste 210. In one form, this vapor steam can be directed to energy generation system 50.

In one form, thermal drying system 3241 includes a steam heated hollow-stem auger 3245 to convey agricultural waste 210 through housing 3244 that bisects hot-box 3243. Steam heated hollow-stem auger 3245 will be flighted with each individual flight of flighted auger section 3246 being designed for optimal conveyance of agricultural waste 210 given the average moisture content of agricultural waste 210 within flighted auger section 3246. As agricultural waste 210 transverses the length of housing 3244, thermal energy will volatilize some water and some volatile organic compounds. These vapors will be drawn under negative pressure into vapor recovery system 3247. It is contemplated that thermal drying system 3241 can be natural gas or steam powered. If thermal drying system 3241 is steam powered, it is contemplated that it may obtain steam directly from energy generation system 50 dependent on the form of energy generation system 50. It should be appreciated that thermal drying system 3241 is only one non-limiting example for drying 3240, and that other processes for drying 3240 are contemplated. Additionally or alternatively, it is contemplated that drying 3240 may be absent from waste processing system 30.

With reference to FIG. 8, wastewater treatment system 40 includes a wastewater treatment facility 401 with primary wastewater treatment 410 and secondary wastewater treatment 420 to treat wastewater 340, 630 from waste processing system 30 and from end-user 60, respectively. It should be appreciated however that wastewater treatment system 40 may utilize treatment processes in addition to or in lieu of those illustrated. Usable water 440 leaving wastewater treatment system 40 can be returned for use by the at least one agricultural facility 20, 120 or provided to secondary facilities 21. It is also contemplated that useable water 440 can be provided to any of the subsystems of systems 10, 110 or to any party having a need for water.

While all types of current wastewater treatment methods are contemplated, FIGS. 8 & 9 illustrate one exemplary form. Secondary wastewater treatment 420 includes an Algaewheel® system available from Algaewheel, Inc. having a mailing address of 1426 West 29^{th} Street, Suite 206, Indianapolis, Ind., 46208. Algaewheel® system 4210 uses fixed and suspended algae and bacterial populations in a symbiotic waste treatment process. Algaewheel® system 4210 uses a single-pass, three-stage treatment train including aerobic, anoxic and anaerobic components to achieve effective biological treatment.

Algaewheel® system 4210 generally removes ammonia and phosphates from wastewaters 340, 630. This efficiency arises from the use of solar energy by the algae. The design of Algaewheel® system 4210 typically provides a much greater efficiency and/or lower operational expenses than traditional wastewater treatment. If produced by energy end-user 60, CO₂ 640 may also be used to promote the growth of algae in the Algaewheel® system 4210. For example, in one form where energy end-user 60 is a manufacturing facility such as biofuel production facility, CO₂ will be produced, further details of which are discussed in connection with FIG. 13. After processing in wastewater treatment system 40, resulting usable water 440 can be returned for use by the at least one agricultural facility 20, 120 in irrigation or provided to secondary facilities 21 to be employed in an efficient aquaculture operation or for other outside water needs. Algae biomass 4211 produced by Algaewheel® 2410 can be added to biomass 330 for consumption by and for energy production with energy generation system 50.

Other forms of the present application contemplate the use of conventional waste water treatment systems such as bacteriological and/or chemical-based treatments in addition to or in lieu of Algaewheel® system 4210.

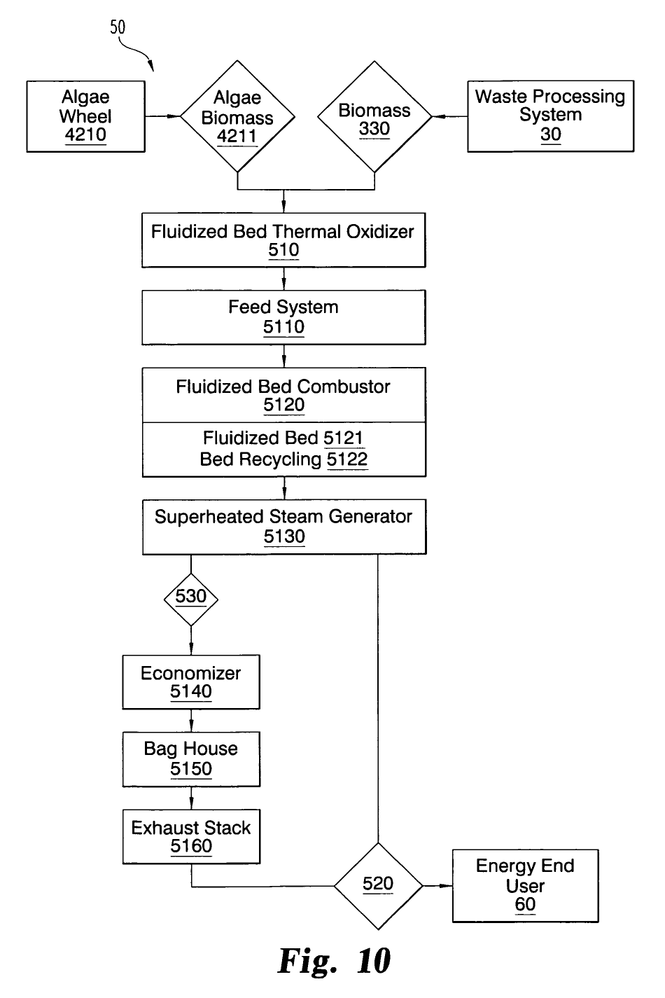

In the form illustrated in FIGS. 10-11, energy generation system 50 includes a fluidized bed thermal oxidizer 510 which is operable to produce energy 520 in the form of steam, although other forms or energy generation system 50 are contemplated. Moreover, it is contemplated that energy generation system 50 could produce energy 520 in forms in addition to or in lieu of steam, such as electricity. One form of the present application contemplates using a fluidized bed thermal oxidizer manufactured by Energy Products of Idaho (EPI). Biomass 330 is conveyed directly into feed system 5110 of fluid bed combustor 5120 to produce steam from a superheated steam generator 5130 and yield exhaust 530 as a by-product. Exhaust 530 can be treated by economizer 5140 and bag house 5150 before exiting exhaust stack 5160.

Fluidized bed combustor 5120 uses a heated bed of sand-like material suspended to create fluidized bed 5121 within a rising column of air to burn many types and classes of fuel. This technique results in improved combustion efficiency of high moisture content fuels, and is adaptable to a variety of “waste type fuels.” The bed material provides a scrubbing action on the fuel particles which enhances the combustion process by stripping away the carbon dioxide and char layers that normally form around the fuel particle. This scrubbing action allows oxygen to reach the combustible material much more readily and increases the rate and efficiency of the combustion process.

Fluid bed combustor 5120 preferably includes uniform bed drawdown, integrated air cooling and automatic cleaning and reinjection of the bed material in bed recycling system 5122. These features enable systems to operate on fuels with significant quantities of 4-inch minus noncombustible tramp material (contaminants such as rocks, metal, etc.). While contemplated, grate style systems sometimes have problems with tramp materials and ash slag can cause problems requiring a shutdown to correct. In other fluidized bed systems, tramp materials can build to the point that fluidization is no longer possible because clinkers are allowed to form. In these systems, a shutdown is usually required to clean out the accumulation. Further details of fluidized bed systems are disclosed in U.S. Pat. Nos. 5,101,742; 5,060,584; 4,510,021; 4,448,134; 4,253,824; 4,075,953; and 4,060,041, which are each incorporated herein by reference in their entirety.

Turbulence in superheated steam generator 5130 combined with the tumultuous scouring effect and thermal inertia of fluidized bed 5121 provide for complete, controlled and uniform combustion. These factors are important to maximizing thermal efficiency, minimizing char, and controlling emissions. The high efficiency of fluidized bed thermal oxidizer 510 makes it particularly well suited for fuels with low BTU value and high moisture characteristics. In typical units, the carbon burnout percentages within superheated steam generator 5130 are well in excess of 99%. Some additional benefits may include: low emission, favorable ash properties, and operating flexibility.

Emissions from exhaust stack 5160 of fluidized bed thermal oxidizer 510 are typically lower than conventional technologies for the following reasons. Low combustion temperatures and low excess air within the bed reduce the formation of certain emissions such as NOx. High combustion efficiency results in flue gases that contain low amounts of CO. Emissions such as SOx and NOx may be abated within the fluidized bed system by injecting limestone into the bed and ammonia into the vapor space.

The high combustion efficiency of fluidized bed thermal oxidizer 510 results in a reduced amount of inorganic material as fine ash. The remaining larger material consists mainly of non-combustibles, such as rocks, and wire brought in with the fuel, and coarse sand-like neutral particles. Low combustion temperatures in the fluidized bed thermal oxidizer 510 minimize the formation of toxic materials that might go into the ash. Therefore, it is contemplated that the resultant ash could be utilized for other purposes such as cement production.

The thermal “flywheel” effect of fluidized bed 5121 allows swings in moisture and heating content of biomass 330 to be absorbed by fluidized bed thermal oxidizer 510 without negative impact. Conversely, the low fuel inventory present in the unit makes it very responsive to varying loads. The fluidized bed thermal oxidizer 510 also maintains efficiency during system turn-down. In one form, it is contemplated that fluidized bed thermal oxidizer 510 reduces maintenance costs and down time. Preferably, fluidized bed thermal oxidizer 510 achieves operating availabilities above 98% in order to keep operating costs relatively low given the difficult fuels they burn.

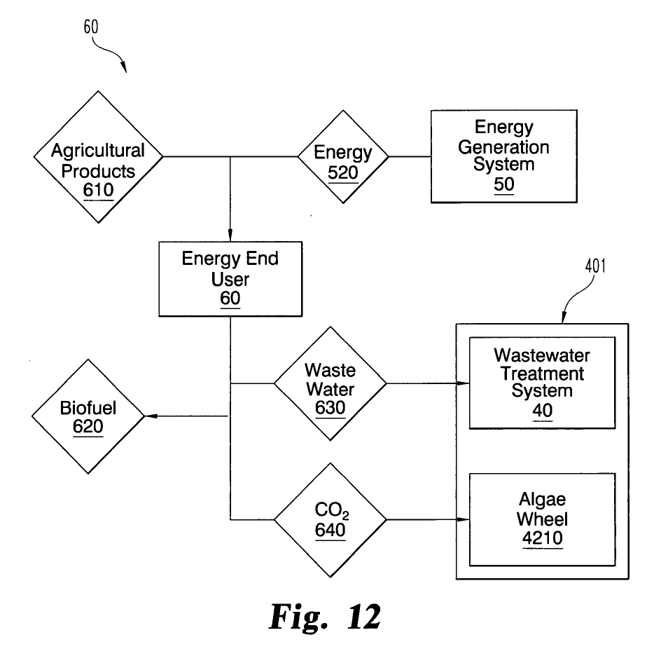

The combustion of biomass 330 and algae biomass 4211 will produce a sufficient quantity of energy 520 in the form of steam to meet the steam needs of energy end-user 60. It should be appreciated that, in alternative forms, energy 520 in the form of electricity could be provided in a quantity sufficient to meet the needs of energy end-user 60. In the embodiment illustrated in FIG. 12, energy end-user 60 is a manufacturing facility in the form of a biofuel production facility which utilizes agricultural products 610 to produce biofuel 620. Wastewater 630 and CO₂ 640 are common by-products generated during the production of biofuel 620, and can be utilized in system 10 as described herein. Fluidized bed thermal oxidizer 510 can produce low pressure saturated steam from superheated steam generator 5130 to meet the needs of energy end-user 60 in the form of a biofuel production facility, or can produce high pressure steam that can be expanded over steam turbines to simultaneously generate electrical power. As indicated above, other systems of energy production are also contemplated. One such system is a closed loop heat recovery turbo-expander system, which converts waste heat to electrical power generation. This system may be less efficient than large steam turbines for power production at high combustion temperatures. However, one such system from WOW Energies, Inc. is far more efficient than steam turbines with lower temperature waste heat sources. As such, it may allow greater overall efficiency when combined with fluidized bed thermal oxidizer 510. It should also be appreciated that the form of energy 520 produced by energy generation system 50 may vary dependent on the needs of end-user 60 of energy 520. For example, in system 110 set forth in FIG. 2, energy 520 is provided to the at least one agricultural facility 120 instead of energy end-user 60, and may be for example, in the form of electricity instead of steam.

In one form, a system includes multiple agricultural facilities which operate to produce agricultural waste; a waste processing system; a waste water treatment system; an energy generation system which operates to generate energy such as steam or electricity; and an energy end-user such as a manufacturing facility like a biofuel production facility that is powered by energy produced by the energy generation system. Agricultural facilities provide agricultural waste to the waste processing system which processes the agricultural waste to produce a biomass. The biomass is subsequently consumed by the energy generation system to produce energy while wastewater produced by the waste processing system is processed in the wastewater treatment system for reuse.

In another form, a method includes transferring agricultural waste from multiple agricultural facilities via vacuum conveyance system to a waste processing system. The agricultural waste is subjected to dewatering and thermal drying processes which create wastewater and biomass. The wastewater is transferred to a wastewater treatment system and the biomass is transferred to an energy generation system which includes a fluidized bed thermal oxidizer. The wastewater is processed by the wastewater treatment system and provided as useable water which can be returned to the agricultural facilities or to other users. In one form, energy such as steam or electricity generated by the fluidized bed thermal oxidizer is used by the agricultural facilities.

In yet another form, a waste management and processing system includes multiple agricultural facilities which operate to produce agricultural waste; a centralized waste pit which operates to receive agricultural waste; a vacuum conveyance system which operates to deliver agricultural waste to a waste processing system; and a biofuel facility which operates to convert agricultural products to produce biofuel. The waste processing system operates to generate a biomass which is used to generate steam to power the biofuel facility.

In still another form, a method includes processing agricultural waste from at least one agricultural facility into a combustible biomass. The biomass is subsequently used to power an energy generation system that provides energy to a biofuel facility.

Any theory, mechanism of operation, proof, or finding stated herein is meant to further enhance understanding of the present invention and is not intended to make the present invention in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the invention, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary. While the invention has been illustrated and described in detail in the drawings and foregoing description, the same is to be considered as illustrative and not restrictive in character, it being understood that only the selected embodiments have been shown and described and that all changes, modifications and equivalents that come within the spirit of the invention as defined herein or by any of the following claims are desired to be protected.

What is claimed is:

1. A system for providing energy from agricultural waste, comprising:
at least one agricultural facility from which said agricultural waste is provided;
a waste processing system coupled with said at least one agricultural facility to receive said agricultural waste, said waste processing system being structured to remove water from said agricultural waste to produce a biomass;
an energy generation system coupled with said waste processing system to receive said biomass, said energy generation system being structured to utilize said biomass to produce a quantity of energy;
at least one energy end-user coupled with said energy generation system to receive and utilize said quantity of energy; and
a wastewater treatment system coupled with said energy end-user and said waste processing system to receive wastewater therefrom and treat said wastewater to provide treated water to at least one of said at least one agricultural facility, said at least one energy end-user, said energy generation system and said waste processing system.

2. The system of claim 1, wherein said at least one energy end-user is a manufacturing facility.

3. The system of claim 2, wherein said manufacturing facility is a biofuel facility.

4. The system of claim 3, wherein said energy is steam and said biofuel facility is structured to utilize said steam to produce a biofuel.

5. The system of claim 1, wherein said at least one agricultural facility includes a plurality of dairies.

6. The system of claim 1, further comprising a vacuum conveyance system structured to deliver said agricultural waste to said waste processing system.

7. The system of claim 6, wherein said vacuum conveyance system includes a valve pit structured to collect said agricultural waste and a main waste line structured to convey said agricultural waste from said valve pit to a vacuum station.

8. The system of claim 1, wherein said waste processing system removes water from said agricultural waste by at least one of homogenization, electro-coagulation, decanting, drying and horizontal screw pressing.

9. The system of claim 1, wherein said waste processing systems performs a drying process that includes electro-coagulation of said agricultural waste.

10. The system of claim 9, wherein said drying process further includes at least one decantation of said agricultural waste.

11. The system of claim 1, wherein said energy generation system includes a fluidized bed thermal oxidizer and said quantity of energy includes steam.

12. The system of claim 11, wherein said fluidized bed thermal oxidizer includes a feed system structured to receive said biomass and feed said biomass into a fluid bed combustor including a fluidized bed and a bed recycle system structured to produce said steam from a superheated steam generator and wherein an economizer, a bag house, and an exhaust stack are structured to remove a portion of pollutants from an emission produced by said fluid bed combustor.

13. The system of claim 1, wherein said wastewater treatment system further comprises a primary wastewater treatment and a secondary wastewater treatment including an Algaewheel® which produces an algae biomass.

14. The system of claim 13, wherein said energy generation system utilizes at least a portion of said algae biomass to produce said quantity of energy.

15. A system, comprising:
at least one agricultural facility operable to produce agricultural waste;
a vacuum conveyance system coupled to said at least one agricultural facility and operable to deliver said agricultural waste to a waste processing system, said waste processing system being operable to remove water from said agricultural waste to provide a biomass;
an energy generation system operable to utilize said biomass to produce energy; and
wherein said energy generation system provides said energy to said at least one agricultural facility.

16. The system of claim 15, wherein said agricultural waste is electro-coagulated and decanted at least once by said waste processing system.

17. The system of claim 16, further comprising a wastewater treatment system.

18. The system of claim 17, wherein said wastewater treatment system is coupled to said waste processing system and operable to treat the water removed from said agricultural waste.

19. A method, comprising:
collecting agricultural waste from at least one agricultural facility;
removing water from said agricultural waste to provide a biomass and a first portion of wastewater;
utilizing said biomass to produce a quantity of energy;
providing said energy to at least one energy end-user;
treating said first portion of wastewater to provide useable water; and
transferring said useable water to said at least one agricultural facility.

20. The method of claim 19, wherein said at least one energy end-user is a manufacturing facility.

21. The method of claim 20, further comprising powering production processes of said manufacturing facility with said quantity of energy, said production processes generating a second portion of wastewater.

22. The method of claim 21, further comprising treating said second portion of wastewater to provide useable water and transferring said useable water to said at least one agricultural facility.

23. The method of claim 21, wherein said manufacturing facility is a biofuel facility, said quantity of energy includes steam and said production processes provide a biofuel.

24. The method of claim 24, wherein said steam is produced by combusting said biomass in a fluidized bed thermal oxidizer.

25. The method of claim 19, wherein removing water from said agricultural waste includes electro-coagulation of said agricultural waste.

26. The method of claim 25, wherein removing water from said agricultural waste further includes decantation of water from said agricultural waste following said electro-coagulation.

27. A method for preparing a biomass, comprising:
providing agricultural waste;
electro-coagulating said agricultural waste to separate solids in said agricultural waste from water in said agricultural waste; and
removing said solids from said agricultural waste to provide said biomass and a quantity of wastewater.

28. The method of claim 27, wherein said removing includes decanting said agricultural waste.

29. The method of claim 27, wherein said removing includes drying said solids to reduce residual water therein.

30. The method of claim 27, wherein said providing includes collecting said agricultural waste with a waste vacuum system from at least one agricultural facility.
